(19) United States
(12) Patent Application Publication
(10) Pub. No.: US 2021/0393861 A1
(43) Pub. Date: Dec. 23, 2021

(54) FLUID AND AIR VOLUME MEASUREMENT SYSTEM FOR A BREAST PUMP ASSEMBLY

(71) Applicant: Willow Innovation, Inc., Mountain View, CA (US)

(72) Inventors: Calmer Mathew, Sacramento, CA (US); Erica Keenan, San Francisco, CA (US); Brian Mason, Lexington, CA (US); John Chang, Los Altos, CA (US); Paul Dietrich, Palo Alto, CA (US); Rory Nordeen, San Francisco, CA (US); Joel Jensen, Redwood City, CA (US); Joshua Makower, Los Altos Hills, CA (US); Edison Yee, Los Altos, CA (US)

(21) Appl. No.: 17/467,312

(22) Filed: Sep. 6, 2021

Related U.S. Application Data
(63) Continuation of application No. PCT/US20/21502, filed on Mar. 6, 2020.
(60) Provisional application No. 62/815,412, filed on Mar. 8, 2019.

(51) Int. Cl. A61M 1/06

(52) U.S. Cl. CPC A61M 1/067; A61M 2205/15; A61M 2205/3379; A61M 2209/088; A61M 2205/3344; A61M 2205/50; A61M 2205/332

(57) ABSTRACT

Systems and methods with variable and customized functionality for pumping milk from a breast and calculating or determining volumes pumped, wherein the milk is expressed from the breast under suction and milk is expulsed from the pumping mechanism to a collection container under positive pressure.

### FIELD OF THE DISCLOSURE

The present disclosure generally relates to measurement systems for a portable breast pump assembly.

### BACKGROUND OF THE DISCLOSURE

As more women become aware that breastfeeding is the best source of nutrition for a baby, and also offers health benefits to the nursing mother, the need is increasing for breast pump solutions that are user-friendly and accurately determine or track pumped milk volumes. This is particularly true for the working mother, who is away from the home for eight to ten hours or more and needs to pump breast milk in order to have it available for her baby, but it is also a requirement for many other situations where the mother is away from the privacy of the home for an extended period, such as during shopping, going out to dinner or other activities.

Although a variety of breast pumps are available, a number are awkward and cumbersome, requiring many parts and assemblies and being difficult to transport. Hand pump varieties that are manually driven are onerous to use and can be inconvenient to use. Some powered breast pumps require an AC power source to plug into during use. Some systems are battery driven, but draw down the battery power fairly rapidly as the motorized pump continuously operates to maintain suction during the milk extraction process.

There is a continuing need for a small, portable, self-powered, energy efficient, wearable breast pump system that accurately calculates or determines pumped volumes, that mimics natural nursing, and is discrete by not exposing the breast of the user and nearly unnoticeable when worn.

To ensure that the nursing baby is receiving adequate nutrition, it is useful to monitor the baby's intake. It would be desirable to provide a breast pump system that easily and accurately monitors the volume of milk pumped by the system, to make it convenient for the nursing mother to know how much milk has been extracted by breast pumping. It would also be desirable to track milk volume pumped per session, so that the volume of milk contained in any particular milk collection container can be readily known.

Moreover, there are needs for approaches to pumping that measure both fluid pumped as well as air that is pumped to thereby enable the system to diagnose an air leak such as from an improper or inadequate latch or device assembly or damage and alert the user into action.

There is thus a continuing need for a breast pump system that is effective and convenient to use. The present disclosure addresses these and other needs.

### SUMMARY OF THE DISCLOSURE

Briefly and in general terms, the present disclosure is directed toward a fluid volume measurement system for a breast pump assembly. The system includes structure and functionality configured to accurately assess pumped volumes in real time. In one embodiment, the system includes breast contacting structure and a collection or storage container or assembly, and structure that delivers milk from a breast to the collection assembly. The method involves pumping milk from a breast and delivering the pumped milk into the collection assembly or storage container. In one particular aspect, the breast pump system responds in real time to optimize pumping action for a particular user during a particular pumping session. The system also provides for manual adjustments to one or more of rate and levels of pumping pressure or suction.

According to one aspect of the present disclosure, the system is configured to assess an internal volume of a closed system pathway or tube segment. In a single sample, volume can be assessed from pump sensors, namely strain gauge measurements and paddle locations in a preferred configuration. Multiple measurements taken with different strain/paddle locations while maintaining a closed system allow for percentage of air and fluid in the internal system to be determined. These volume measurements are taken in the closed system pathway or tube segment at any time during a pumping session. When taken before and after a purge, the difference between measurements enable the total volume purged to be determined. A combination of multiple measurements each before and after purges enable the determination of total volume of air expelled and total volume of fluid expelled in a purge. The system further includes a non-transitory computer readable medium having stored thereon instructions executable by a computing device to cause the computing devices to perform functions associated with and directed by the instructions.

Moreover, in one aspect, analyzing data from multiple purges in succession allows for continuous air leaks to be detected, and accurate cumulative volume of air and fluid pumped into the milk receptacle to be calculated such as is particularly relevant to a closed system. Air leaks are also detected outside of a purge by taking multiple measurements with different strain/paddle locations in the closed system pathway or tube segment, at any time during a pumping session.

In yet another aspect, air leaks are identified and calculated by taking two measurements of a volume-map code while a pinch foot is open and the system is pumping, and while also taking two measurements of vacuum levels. A dVolume/dVacuum relationship is generated to measurement volume changes over vacuums and to thus recognize and/or assess the existence and magnitude of an air leak.

In further aspects, accurate mapping of sensor data to internal tube volume is employed. Thus, when the system is closed, an accurate estimate of the internal volume of that system from readily available sensor data is built and utilized. Learnings about the pump system facilitate improved and more accurate sensor readings, namely how measurements must be constrained in order to produce an accurate volume and how the system is manipulated to create such readings. Volume measurements are used in novel ways to determine air volume and fluid volume in the closed system segment or pathway at any moment, and hence, over time, facilitate determining ratio of air to fluid and how much has been pushed into a collection receptacle.

In one or more embodiments, the system includes a controller that accomplishes real time pressure control inside the system. In a particular approach, such pressure control can be accomplished via a force gauge or pressure or other sensor. In one or more embodiments, the system includes a controller providing automated compliance sensing and response. In one or more embodiments, the system includes one or more controllers that automatically detects one or more of letdown, overfill and flow.

According to another aspect of the present disclosure, a method of operating a system for pumping milk includes or involves one or more of: providing the system comprising a skin contact member configured to form a seal with the breast, a conduit in fluid communication with and connected to the skin contact member; a driving mechanism including a compression member configured to compress and allow decompression of the conduit in response to inward and outward movements of the compression member, a sensor, and a controller configured to control operation of the driving mechanism; sealing the skin contact member to the breast; operating the driving mechanism to generate predetermined pressure cycles within the conduit; monitoring by the controller of at least one of position and speed of movement of the compression member relative to the conduit; measuring or calculating pressure within the conduit; maintaining or modifying motion of the compression member as needed, based upon feedback from the calculated pressure and at least one of force, position and speed of movement of the compression member, to ensure that the predetermined pressure cycles continue to be generated; and calculating volumes pumped via strain gauge measurement and paddle location.

These and other features of the disclosure will become apparent to those persons skilled in the art upon reading the details of the systems and methods as more fully described below.

### BRIEF DESCRIPTION OF THE DRAWINGS

FIG. 1A shows a perspective view of a breast pump system according to an embodiment of the present disclosure.

FIG. 1B is a rear view, depicting the flange of the pump system of FIG. 1A.

FIG. 2 shows a front view of the system of FIG. 1 with the shell removed.

FIG. 3 depicts a back view of the system of FIG. 1 with the flange removed.

FIG. 4 is a cross-sectional side view of the system of FIG. 1.

FIG. 5 is an inside view of the system of FIG. 1, depicting the flex conduit of the pump assembly.

FIG. 6 is an exploded view of the system of FIG. 1, depicting mechanical components of the system.

FIG. 7 is a schematic representation, depicting operational components of the system.

FIG. 8 is a flowchart, depicting one approach to a volume determination.

FIG. 9A is a graphical representation, depicting a pumping waveform.

FIG. 9B is a graphical representation, depicting data associated with an operating pump.

FIG. 10 is a top view, depicting one embodiment of a storage collection assembly of the present disclosure.

FIG. 11 is an enlarged view, depicting an end of the storage collection assembly of FIG. 10.

FIG. 12 is an enlarged view, depicting a valve assembly of the storage collection assembly.

FIG. 13 is a perspective view, depicting a storage collection assembly connected to the system.

### DETAILED DESCRIPTION OF THE DISCLOSURE

Before the present systems and methods are described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the disclosure. The upper and lower limits of these smaller ranges may independently be included or excluded in the range, and each range where either, neither or both limits are included in the smaller ranges is also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the present disclosure, the preferred methods and materials are now described. All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. Thus, for example, reference to “a sensor” includes a plurality of such sensors and reference to “the pump” includes reference to one or more pumps and equivalents thereof known to those skilled in the art, and so forth.

The publications discussed herein are provided solely for their disclosure prior to the filing date of the present application. The dates of publication provided may be different from the actual publication dates which may need to be independently confirmed.

Various details of related systems can be found in U.S. application Ser. No. 15/083,571 (now U.S. Pat. No. 9,539,376), Ser. Nos. 15/361,974; 15/362,920; and 15/406,923 (now U.S. Pat. No. 10,434,228) each filed Jul. 21, 2015, and Ser. No. 16/050,201 filed Jul. 31, 2018, each of which are hereby incorporated herein, in their entireties, by reference thereto.

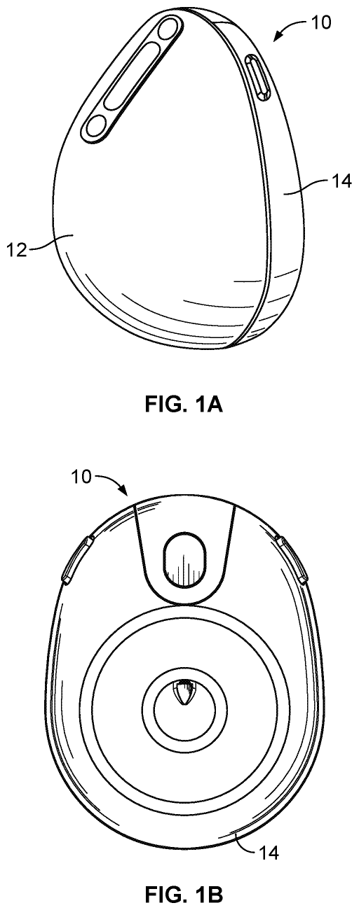

FIGS. 1A-B are perspective and back views of a breast pump system 10 according to an embodiment of the present disclosure. The breast pump system 10 can include one or more of the below introduced or described features or functions, or a combination thereof. The housing or outer shell 12 of system 10 can be shaped and configured to be contoured to the breast of a user and to thus provide a more natural appearance when under the clothing of the user. As can be appreciated from the figures, the system can define a natural breast profile. The natural breast profile is contemplated to fit comfortably and conveniently into a bra of a user and to present a natural look. As such, the profile is characterized by having a non-circular base. Extending from the base are curved surfaces having asymmetric patterns. Moreover, like natural breasts, the profile of the device or system is contemplated to define one or more asymmetric curves and off-center inertial centers. Various natural breast shapes can be provided to choose from to the tastes and needs of a user. An opposite side of the pump system 10 is configured with a flange 14 which is sized and shaped to engage a breast of a user. The flange 14 is contoured to comfortably fit against a wide range of user's bodies and to provide structure for sealingly engaging with breast tissue. In one particular embodiment, the flange 14 can form generally rigid structure, and alternatively or additionally unlike a standard flange can lack sharp edges or a lip portion against which breast tissue might be engaged during use. In this regard, the flange includes surfaces that extend outwardly from a nipple receiving portion of the flange to engage breast tissue, thus providing extra surface area for comfortably contacting tissue.

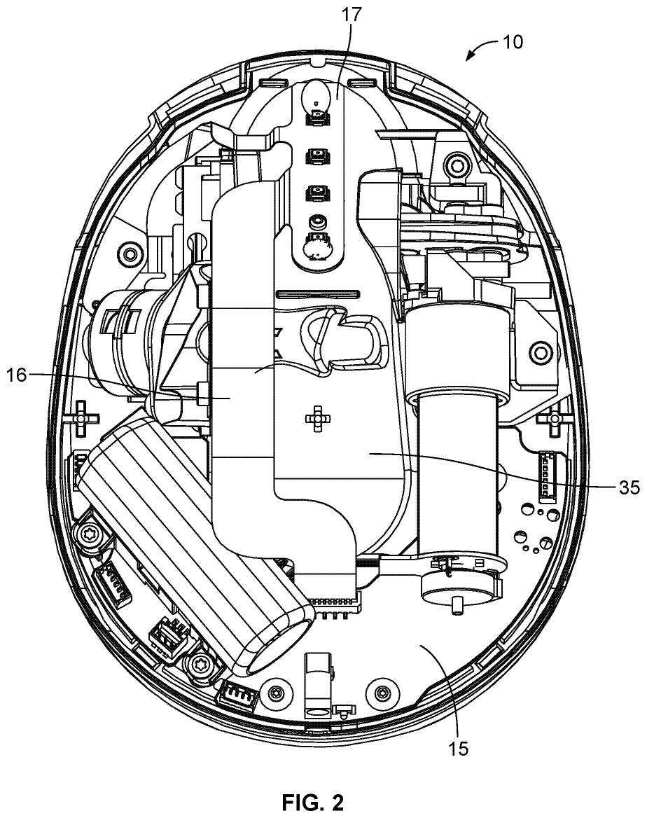

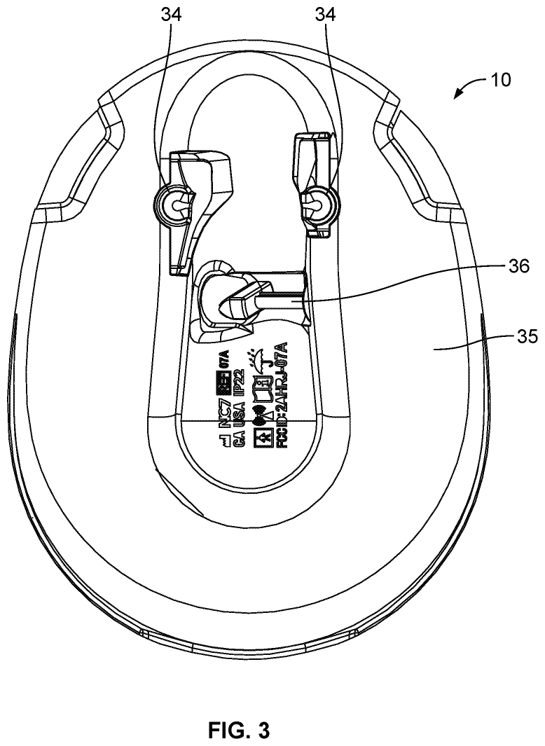

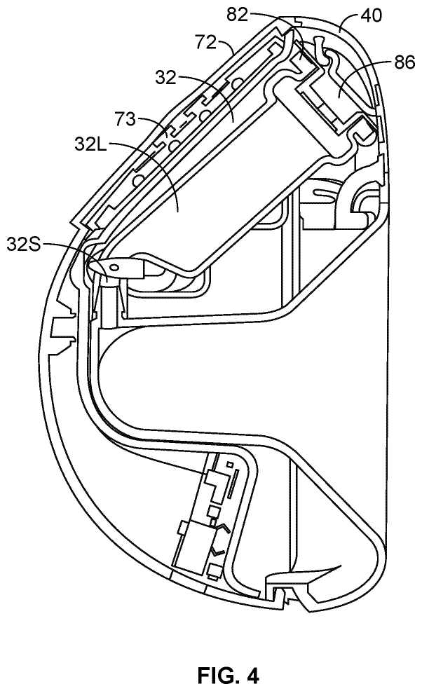

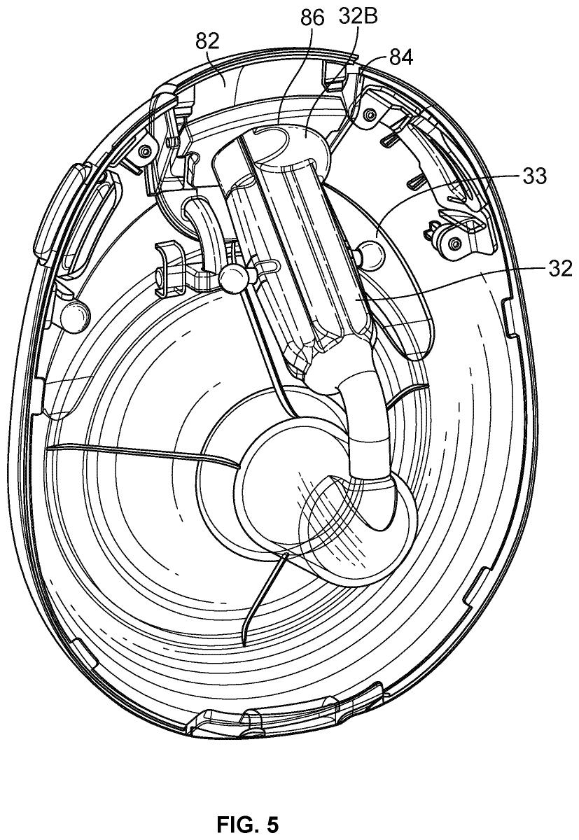

FIG. 2 is a front view of the system 10 of FIG. 1, with the housing or outer shell 12 having been removed and made transparent to show components otherwise covered by the housing 12. In particular, with the housing 12 removed, various electronic components can be identified. The system controller is embodied in a circuit board 15 that is in communication with a flex-circuit 16, each cooperating to connect to and control various electro-mechanical components of the system 10. A control panel 17 is in electronic communication with the controller via the flex-circuit 16 and provides the user with the ability to power the system on and off as well as to alter functioning. One or more motors 44, 46 are further provided and controlled electronically by the system to effect manipulation of actuators (described below) operating on a conduit or flex-tube 32 (See FIGS. 4 and 5). A battery 48 is included to provide a rechargeable power source and is configured to be plugged into a power source for charging. Further, there is provided a load cell assembly 54 that is configured to provide a pressure sensing function as described below. It is contemplated that at least in one embodiment, the conduit or flex-tube 32 is oriented to run from inferior to superior relative to the nipple of a breast when the user is upright.

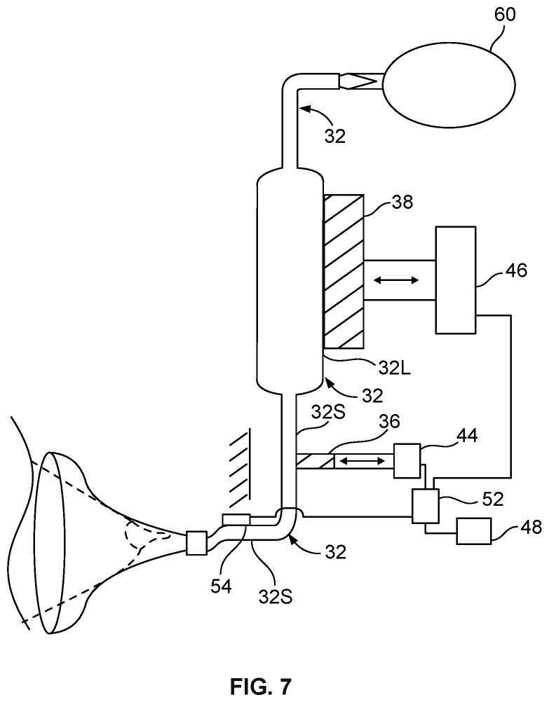

FIG. 3 shows an opposite side of the system 10 with the flange 14 removed to illustrate more details of the pumping function. The conduit or flex-tube 32 (See FIGS. 4-6) includes generally spherically shaped connectors 33 that are sized and shaped to be removably received in recesses 34 formed in a pump chassis 35. The connectors 33 are designed to automatically engage with grooves in the pump linked to a moving motor paddle and a strain gauge without the user being aware or having to make adjustments, or assemble parts. The pump chassis 35 functions to support the electronic and electro-mechanical structures of the system 10 (See also FIG. 2). It also provides spacing for a pinching actuator 36 that is configured to be advanced and retracted toward and away from the conduit or flex-tube 32 as described further below. Other pumping action is accomplished through the engagement of the conduit or flex-tube 32 with recesses 34 by a compression and expansion member 38 (See FIG. 7).

In general, real-time pressure control can be managed by a controller of the system 10. The controller tracks pressure and moves a pump motor either in or out to influence the pressure in the direction of its choosing. By way of oscillating motion of the motor, the pump can be configured to pull on the connectors 33 of the conduit or flex-tube 32 structure to increase its volume. If there is vacuum in the system 10 that vacuum can be increased as the volume of the tube increases. Pushing in the tube decreases its volume. This in turn causes the vacuum level to decrease in the tube, and can cause a relative positive pressure if vacuum decreases enough. The pump controller applies these principles, sensing the current pressure and then nudging a compression member or paddle of the motor assembly in a direction required to generate a pressure target. By doing this repeatedly in real time, the system can create a controlled vacuum waveform that matches waveforms desired to be applied to a user's nipple.

The pump can slowly pull the compression member or paddle out until it hits a pre-determined target. Should the paddle be moved to the end of its range without being able to generate a desired vacuum, the system will be purged to generate more vacuum potential. The purge functions to push material out of the system to create a strong vacuum potential. It accomplishes this by first closing a pinch on the conduit or flex-tube or closing off the flex-tube with a flap, dam, etc., then evacuating the flex-tube, for example, by pushing closed the paddle, which forces volume out of the flex-tube and any fluid or air that was inside that volume is also ejected through the one-way valve and into the collection receptacle. When the paddle retracts again, it can then generate much higher vacuum as contents of the tube had been previously purged. Once a higher vacuum can be generated, the system can open the pinch valve so that the desired vacuum profile can be applied to a breast and desired pressure waveform can be produced.

When the system is filled with air, it is very compliant such that a large change in motor positioning makes only a small change in vacuum. When the system is filled with fluid on the other hand, a small change in motor positioning makes a big change in vacuum. In one particular approach, an encoder including a plurality of spaced magnets is associated with the motor. The magnets can be placed along a periphery of a generally disc shaped encoder with the magnets oriented parallel to the axis of rotation of the encoder. One or more hall effect sensors can be configured on or surface mounted to the circuit board 15 and positioned to read the motion and position of the magnets. In this way, the position of the motor can be determined and monitored. Thus, a challenge can be to configure the system so that it is stable when the system is responsive, and effective when it is not as responsive. One contemplated approach is to tune the controller for a relatively rigid system and to input unit-less quantities that move the motor in required directions where the amplitude of which is modified depending on the output of the system. Accordingly, a cascade controller can be created to grow an input wave if system output is smaller than desired to hit pressure targets and can be shrunk if the system output is larger than required. This can be accomplished in real time by observing output verses input. In this way, the controller can be continuously adjusting target waveforms. Top half and bottom half waveforms can have independent control which facilitates centering waveforms in an effective manner, and results in a system that is both very accurate and quick to adjust.

The system can further be provided with automated letdown detection. The pump can sense when it is full of fluid and responds accordingly by switching between pumping and letdown when fluid has begun to flow. In one approach an algorithm incorporated into the system can operate to look at the ratio of maximum and minimum of a target wave in the pump and compare that against the output of the pump. The result is a unit-less but very reliable sensing of system compliance. This can be tuned to trigger an internal event when the compliance crosses some known values that represent when the system is full of fluid. Any other measurement of compliance can be used in an equivalent way.

In another approach to letdown detection, it is noted that pushing a tube of air does not generate the same forces as pushing a tube of fluid. Tracking the force generated during a purge can also give a strong indication of when the system is full of fluid. An event can be generated to track this such that when the force of a purge crosses some known threshold the system can be said to be full of fluid rather than air. This approach may involve less tracking of data and less tuning that is subject to change with pump design or breast tissue. In yet another approach, letdown detection can be based upon tracking flow. That is, when flow begins, letdown must have occurred and when a small volume of flow has been collected the system can switch to pumping. Further, letdown can be tracked by looking at the relative rate of change of vacuum measured to motor position. Note that this relative rate of change is a measurement of compliance. As this ratio goes up in magnitude, it can be concluded that the system is filling with fluid.

In yet another approach, a letdown sensing methodology is incorporated into the system so that letdown when about 2.5 ml of milk is detected. Thus, the system changes out of stimulation mode on timing associated with when the mother expresses milk. Accordingly, once the system detects milk is flowing, the same is treated as letdown detection. Later, when the system senses it is full of fluid, there is provided a separate gate-way that allows access to all pump levels.

The sensing mechanism involves looking to see if there are two purges in a short amount of time. In one approach, the system controller determines there is letdown when two purges occur within 45 seconds of each other, and after the seventh second of the session (although such constants can be changed). Basically, if milk is flowing, there will be a couple of purges on the pump without a long wait between them. The time limits imposed help to ensure that a very slow air leak, or some physical adjustment that causes a purge don't trigger the detection. The small time delay (e.g. seven seconds) at the start of the session before the system commences protects against an occurrence of a pump not quite having enough vacuum right when a session starts and needing to do a purge at the start to remedy that.

In one implementation, the system reduces its pumping frequency when letdown detection occurs. This can also be displayed in the system App that the pump is in “expression” mode rather than “stimulation”. Further, the system increases vacuum automatically when all vacuums can be reached and an alert is sent to the system App when all vacuum levels can be reached. The user is thus aided in that she knows that vacuum can be increased, that she has full control over the pumping and is provided with a clear milestone that can be used as a test for proper alignment. That is, not being able to hit such targets in an expected time is used as feedback that a user might need to realign.

Therefore, with this approach, the system can be more responsive as users can sense their own letdown occurring and can reduce anxiety for users having trouble sensing their letdown. Moreover, there can be faster milk collection, removes a need for certain users to lean back to achieve letdown, and reduces the need for the user to constantly monitor the mobile App at the start of pumping.

FIG. 4 illustrates a cross-section of components of a system 10 according to an embodiment of the present disclosure. Flex-tube or conduit 32 (isolated in FIG. 5) includes a large conduit portion 32L that is relatively larger in cross-sectional inside area than the cross-sectional inside area of small conduit portion 32S. The large conduit portion 32L terminates with an opening sized for cleaning and is generally sized to accept a small finger tip. Although both portions 32S and 32L are shown as tubular portions, the present disclosure is not limited to such, as one or both portions could be shaped otherwise. When tubular, the cross-sections may be oval, square, other polyhedral shape, non-symmetrical, or non-geometric shape. Further, the flex-tube 32 can include an enlarged bulbous portion 32B configured near a terminal end of the large conduit portion 32L that is provided to help accommodate system hysteresis.

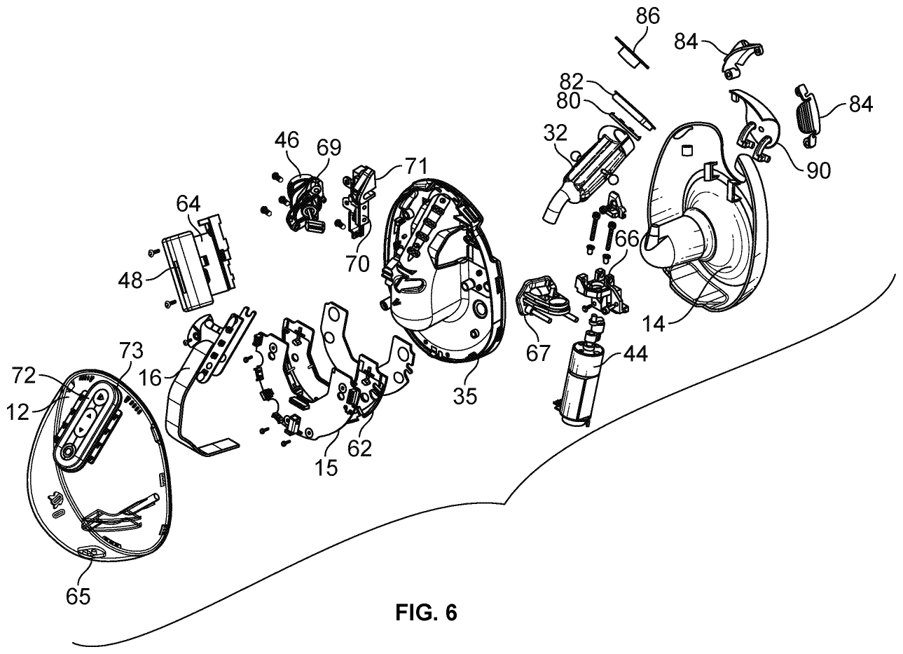

FIG. 6 depicts an exploded view of structural and mechanical components of the system 10. Configured between the housing 12 and flange 14 is the chassis 35. Notably, the chassis can be configured to snap into engagement with the housing 12. Moreover, in a preferred embodiment, the chassis 35 supports directly or indirectly all of the pump components. In particular, a PCB controller mount 62 is supported by the chassis 35 and is configured to be connected to and support the circuit board 15 (See also FIG. 2). A battery bracket 64 is also supported by the chassis 35 and is sized and shaped to receive a rechargeable battery 48 assembly that powers the system 10. A cover jack or power cover 65 is further included to provide access to a reset button charging port for the battery assembly and for accepting a power cord connector (not shown). Motor mounting 66 and motor receiver structure 67 is also supported by the chassis 35 and are configured to receive and support the system motor which is powered by the battery and which functions to move motor operating on the conduit or flex-tube 32. Also supported by the chassis 35 are an actuator bracket 69 that supports the actuator to allow for pinching of the foot on the flextube, and a load cell bracket 70 and load cell receiver 71. Moreover, user interface panel can include a button membrane 72 and a button membrane housing 73 each supported on the housing 12 and placed in engagement with the flex-circuit 16 that provides the user with system control.

In order to connect the conduit or flex-tube assembly 32 to the system 10, there are provided a flex-tube assembly 82. The flex-tube assembly 82 is sized and shaped to be received into slots 84 on the flange. A fluid container fitment 86 (shown in isolation from the container) is sized and shaped to be received into the flex-tube assembly 82. A door assembly 90 is attached to the flange 14 and configured to swing open and closed to both provide access to an interior of the system 10 as well as to support a robust connection between the fitment 86 and flex-tube assembly 82. Accordingly, it is contemplated that in at least one embodiment, the collection or container assembly is supported and maintained in attachment by friction around a shaft of the conduit to the collection or container assembly, and partially by the door assembly 90 which can enclose and hold the collection or container assembly in place. In alternative embodiments, the breast pump assembly can omit a door assembly entirely. Thus, the flange itself can include structure for retaining the container assembly in place. Moreover, the door assembly or other structure that replaces the door assembly can be transparent so that a direct view to the container assembly is provided.

As shown schematically in FIG. 7, latching, pumping and extraction forces can be established by two compression members 36, 38 that are actively driven by motor drivers 44 and 46 respectively. Although more than two compression members could be used and one or more than two drivers could be used, the currently preferred embodiment uses two compression members respectively driven by two drivers as shown. A system controller or system software and/or firmware controls the action of the drivers in real time, responsive to pre-determined latching and production targets or schemes as detected by the pressure sensor or load cell assembly. The firmware can be written so that such targets can be approached at various speeds, sometimes relatively quickly and other times more slowly or gently to thereby provide multiple stimulation and expression levels. Thus, for example, latch can be achieved taking alternatively more gradual or quicker approaches, and there can be controls determining the level at which latch is achieved in order to mimic sucking patterns of a baby.

Various levels of suction can be present during expression as well. Tubing portions 32S and 32L can be closed off, or substantially closed off by compression members 36 and 38, respectively. Moreover, such active pumping members can be configured to engage upon a tubing channel generally perpendicularly to the net flow of fluid or milk within the channel. Also, a pinch region of the tubing channel can be configured to open through passive recoil located next to a compression region of the tubing channel which opens through an assistive active support. Upon powering up the system 10 the compression member 36 opens and the compression member 38 begins to withdraw away and through its connection to structure such as the ball connector of the conduit or flex-tube 32 thereby gradually increases the suction level within tubing 32. When a predetermined maximum suction level is achieved (as confirmed by pressure readings taken from a pressure sensor, described below), the compression member 38 ceases its travel in the current direction, and either maintains that position for a predetermined period of time (or moves slightly in the same direction to compensate for decreasing suction as milk enters the system) when the operating mode of the system 10 has a predetermined time to maintain maximum suction, or reverses direction and compresses the tube 32L until the latch suction level is achieved. Such predetermined levels can be determined employing a test set-up arrangement separate from the pump. If the maximum suction level has not yet been achieved by the time that the compression member can be fully retracted away on the first stroke, then the compression member 36 again compresses the tube 32S to seal off the current vacuum level in the environment of the breast, and the compression member 38 fully compresses the tube portion 32L to squeeze more air out of the system. Then the compression member 36 reopens to fully open tube portion 32S and compression member carries out another stroke, again moving away to generate a greater suction level. This cycling continues until the maximum suction level is achieved. It is noted that it is possible in some cases to achieve the maximum suction level on the first stroke, whereas in other cases, multiple strokes may be required.

Upon achieving the maximum suction, the system may be designed and programmed so that the compression member 38 does not travel to its fullest possible extent in either direction to achieve the maximum and latch suction levels, so as to allow some reserve suction and pressure producing capability. When the maximum suction level has been achieved, and the pumping profile can return to latch vacuum, the compression member 38 advances compressing tubing portion 32L, thereby raising the vacuum in the tubing 32. Upon achievement of the latch suction vacuum, compression member 36 closes off the tubing 32S again to ensure that the latch vacuum is maintained against the breast, so that sufficient suction is maintained. At this stage, the compression member 38 again begins moving away to increase the suction level back to a target suction (such as close to latch vacuum), and compression member 36 opens to allow tube 32S to open and the breast 2 to be exposed to the maximum suction. Alternatively, the system may be programmed so that the compression member 38 cycles between maximum and latch suction levels without the compression member 36 closing during a point in each cycle, with the compression member 36 closing when the latch vacuum is exceeded.

Upon commencing milk extraction, the compression member 36 and compression member 38 can function in the same manner as in latching, but in a manner that follows an extraction waveform determined by the selected extraction pumping determined in real time by system controls which are responsive to the load cell assembly or pressure sensing assembly. At this stage, any sounds created by the pumping action of the system are decreased as milk or fluid flows through the pump mechanism. During the compression stroke of compression member 38, compression member 36 closes when the latch pressure/suction level is achieved. Continued compression by the compression member 38 increases the pressure in the tubing 32 downstream of the compression member 36 to establish a positive pressure to drive the contents (milk) of tube portion 32L out of the tube portion 32L through smaller tubing portion 32S2 downstream of 32L and out through a one-way valve. The positive pressure attained is sufficient to open the one-way valve for delivery of the milk out of the tubing 32 and into a milk collection container. In one embodiment, the positive pressure is in the range of 20 mm Hg to 40 mm Hg, typically about 25 mm Hg. Upon reversing the motion of compression member 38, compression member 36 opens when the suction level returns to the latch suction level and compression member 38 continues to open to increase the suction level to the maximum suction level.

The present disclosure can establish a latch vacuum to cause the flange or skin contact member/breast 14 to seal to the breast. The latch vacuum established by the system is currently about 60 mmHg, but can be any value in a range from about 20 mmHg to about 100 mmHg. Once the system 10 has been latched to the breast via skin contact member 14, the system then cycles between the latch vacuum and a target (also referred to as “peak” or “maximum”) suction level. Due to the fact that the system 10 does not cycle down to 0 mmHg, but maintains suction applied to the breast, with the minimum end of the suction cycle being the latch suction level (e.g., about 60 mm Hg), the nipple does not contract as much as it would with use of a prior art breast pump system. It has been observed that the nipple draws into the skin attachment member 10 with the initial latch achievement in an analogous fashion as the formation of a teat during breastfeeding. Once the vacuum cycles between the latch and target vacuum levels, there is significantly less motion of the nipple back and forth with the vacuum changes, as compared to what occurs with use of prior art systems. The nipple motion (distance between fully extended and fully retracted) during use of the present system is typically less than about 2 mm, and in some cases less than about 1 mm. Accordingly, the system provides latching that is not only more like natural nursing, but the reduced nipple motion is also more like natural nursing as evidenced by scientific literature. In one particular approach, the system can employ ultrasound to observe nipple motion during pumping to ensure that desired nipple motion is achieved.

In one embodiment, the total system volume is about 24.0 cc. The total volume is calculated as the space in the nipple receiving portion (that is not occupied by the nipple) and tube portions 32S, 32L and 32S2 up to the milk collection or container assembly. In the embodiment with total system volume of about 24.0 cc, the active pump volume, i.e., the volume displacement achievable by compressing tube portion 32L from fully uncompressed to the limit of compression by compression member 38 is about 3.4 cc. When there is only air in the tubing 32 of the system 10, pressure swing by moving the compression member 38 inwardly against the tubing portion 32L and outwardly away from the tubing portion is limited, due to the compressibility of the air. In this embodiment, with the system under vacuum of −60 mmHg, a full stroke of the compression member (from compressed to fully uncompressed tube portion 32L) increases the vacuum to −160 mmHg. The ratio of pumping volume to total system volume can be important with regard to power and size of the pumping system. In this embodiment, the tube portion 32L was made of silicone. It has been recognized that reduced motion of the compression members when pumping allows for more quiet action of the pump motor, and a more quiet system overall. Further, the present system employs the milk expressed as the medium for system hydraulics, and this medium is in direct contact with the user's breast against which a vacuum is drawn. Thus, the system can employ air suction against the breast for initial latching and pumping and then converts to utilize expressed breast milk for pumping action or power.

During let down operation, the system 10 operates to effect let down of the milk in the breast, prior to extraction, with a maximum suction target of up to 120 mmHg (typically, about 100 mmHg (−100 mmHg pressure)) to establish let down. The goal of letdown (or non-nutritive suction) is to stimulate the breast to express milk. The relatively shallow (small vacuum change range) and relatively fast frequency of the pumping during this phase are meant to mimic the initial suckling action of a child at the breast. This is because during let down phase, the suction pressure is not allowed to exceed the maximum let down suction of 110 mmHg or 120 mmHg, or whatever the maximum let down suction is set at. Therefore, as the compression member 38 is drawn in a direction away from the tube portion 32L, the system 10 is designed to reach −100 mmHg (a suction pressure of 100 mmHg) (or −120 mmHg, or whatever the maximum let down suction is designed to be), by the time that the compression member 38 has reached a position in which tube 32L is mostly uncompressed.

Subtle variation to pumping can be incorporated into the system to both enhance milk production and to mimic natural nursing. Such variations can be tracked by the system and analyzed to determine which variations are most effective to achieve desired or optimum milk production. To mimic natural nursing, waveform/shape, pumping frequency, amplitude, compression/release, and speed of suction can be varied. This variation can additionally make the breast pump feel more comfortable to the user. In one approach, subtle variations to frequency, amplitude, waveform shape and other parameters can be made throughout pumping so that each period or cycle is different from the last. Alternatively, variation can come at key intervals such after a specific time period or pumping event or on specific cues. Moreover, variations can be random or intentional and by design such as a specific pattern designed to stimulate the most milk production that repeats over the course of a few seconds or minutes. Also, variation can be selected by the user to enhance comfort and/or output and/or system quietness, and separate profiles or settings can be provided to users through user input or system firmware. In one particular aspect, the pump is configured to generate a varying vacuum in a repeating waveform from low vacuum to a higher vacuum then returning to the low vacuum. The waveform period is divided into sections of specified duration and there can be one section with a duration of the waveform period. Where there are multiple sections, the sum of each section duration must/can equal the waveform period and the vacuum for each section is specified by a mathematic function, to thereby provide control of the rate of vacuum change when increasing and decreasing vacuum.

During let down (non-nutritive) the system software and/or firmware communicates instructions to system motors based upon readings taken and communicated from the pressure sensing assembly so that the system is configured to operate between −60 mmHg and −100 mmHg in one example. In this example, the compression member 38 can compress the tubing portion 32L nearly fully and then be moved away from the tubing portion 32L to generate vacuum. The maximum latch suction pressure of −100 mmHg will be reached with a small amount of rebound of the tubing portion 32L and the compression member 38 can be cycled relative to the tubing portion 32L between −100 mmHg and −60 mmHg in a narrow range or band near full compression of the tube portion 32L. As milk flows, that narrow band shifts at which point the tube portion 32L will be purged by fully compressing it to drive out the contents and thereby regain more capacity for pumping with relatively less compression of the tube portion 32L again.

The system 10 is responsive to pressure changes within the tubing 32 caused by entry of milk into the tubing 32. Referring again to FIG. 7, the compression elements 36 and 38 are operatively connected to a driver 44, 46, respectively, for independent, but coordinated driving and retraction of the compression elements 36, 38. When electrically-powered drivers are used, a battery 48 is electrically connected to the drivers 44, 46, as well as the controller 52 and pressure sensor 54, and supplies the power necessary to operate the drivers 44, 46 to drive the compression and retraction of the compression elements 36, 38.

The sensor 54 is used to provide feedback to the controller 52 for controlling the pumping cycles to achieve and/or maintain desired vacuum levels. Sensor 54 is preferred to be a load cell sensor providing data utilized to calculate system pressure, but could also be a pressure, flow, temperature, proximity, motion sensor or other sensor capable of providing information usable to monitor the safety or function of the pump mechanism of system 10. As shown, sensor 54 is a non-contact sensor 54, meaning that it is not in fluid communication with the milk or vacuum space of the system 10.

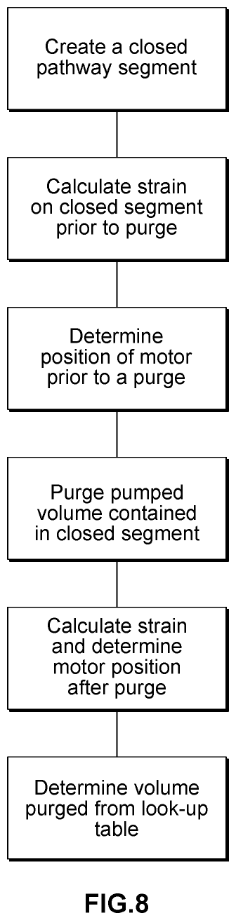

As described above, the conduit or flex-tube 32 is placed in operative connection with a motor. An opposite end of the flex-tube 32 is associated with the sensor 54 that takes the form of a load cell or strain gauge. The positioning of the motor is tracked for example by a sensor, and the force on the tube 32 is assessed to determine volumes pumped using system firmware (See FIG. 8). That is, in a single sample, volume can be assessed from pump sensors, namely strain gauge measurements and paddle or compression element 38 locations. Multiple measurements taken with different strain/paddle locations while maintaining a closed system allow for percentage of air and fluid in the internal system to be determined. These volume measurements are taken in the closed system pathway or tube segment at any time during a pumping session. A closed system pathway or tube segment is created when the flex-tube 32 is pinched by the compression element 36, and a one-way valve leading to the container assembly (described below) is closed. When taken before and after a purge, the difference between volume measurements enable the total volume purged to be determined. A combination of multiple measurements each before and after purge enables the determination of total volume of air expelled and total volume of fluid expelled in a purge. The system is configured to purge early when needed so that vacuums needed can be pulled to get a good measurement after a pinch has closed.

Analyzing data from multiple purges in succession allows for continuous air leaks to be detected, and accurate cumulative volume of air and fluid pumped into the milk receptacle to be calculated. Air leaks are also detected outside of a purge by taking multiple measurements with different strain/paddle locations in the closed system pathway or tube, at any time during a pumping session.

Accurate mapping of sensor data to internal tube volume is employed to determine pumped volumes. When the system is closed, an accurate estimate of the internal volume of that system from readily available sensor data is built and utilized. In one approach, comprehensive data is collected to build a look-up table to derive volumes. Learnings about the pump system facilitate improved and more accurate sensor readings, namely how measurements must be constrained in order to produce an accurate volume and how the system is manipulated to create such readings. Volume measurements are used to determine air volume and fluid volume in the closed system segment or pathway at any moment, and hence, over time, facilitate determining ratio of air to fluid and how much has been pushed into a collection receptacle.

Accordingly, in one preferred embodiment interpreting motor positioning and strain gauge tracking compensates for system noise and hysteresis such as from motor backlash and other mechanical component interactions and engagements, to arrive at a volume calculation. More specifically, a map is created and through polynomial regression a relationship between motor position (i.e. paddle or compression element 38) and tubing strain is made to volumes pumped by the system. System firmware is configured to automatically calculate and track volumes pumped by tracking motor position and tubing strain and correlating this data with the map of volumes pumped results in accurate volume determinations. In this regard, the system 10 includes or communicates with a non-transitory computer readable medium having stored thereon instructions executable by a computing device of the system or external to the system to cause the computing devices to perform functions associated with and directed by the firmware.

Such an approach is not reliant upon a number of variables that may be introduced or inherent in a pumping system. That is, one or more of variables associated with different milk containers, different loading of containers, incoming flow rates, vacuum levels or frequencies, different and random waveforms/shapes, realignments during pumping or air leaks do not have or have a minimum effect on volume determinations.

In another approach to system monitoring, a map relating volume to load cell (force) and motor location is employed. This map is used differentially, that is, the values returned on a per-sample basis are offset by an unknown constant. However, subtracting one measurement from another to look at the difference between measurements allows for a meaningful volume difference in the flextube between the two measurements to be known. By taking this approach, more precision and accuracy is achieved across varying pump input waveforms, frequencies and amplitudes.

Here, the volume map is used regularly while pumping, and volume data is taken in conjunction with vacuum data. By doing so at least two samples in a waveform, each with volume and vacuum data, a data stream is built that represent:

$$\frac{change in volume}{change in vacuum}$$

or its reciprocal:

$$\frac{change in vacuum}{change in volume} .$$

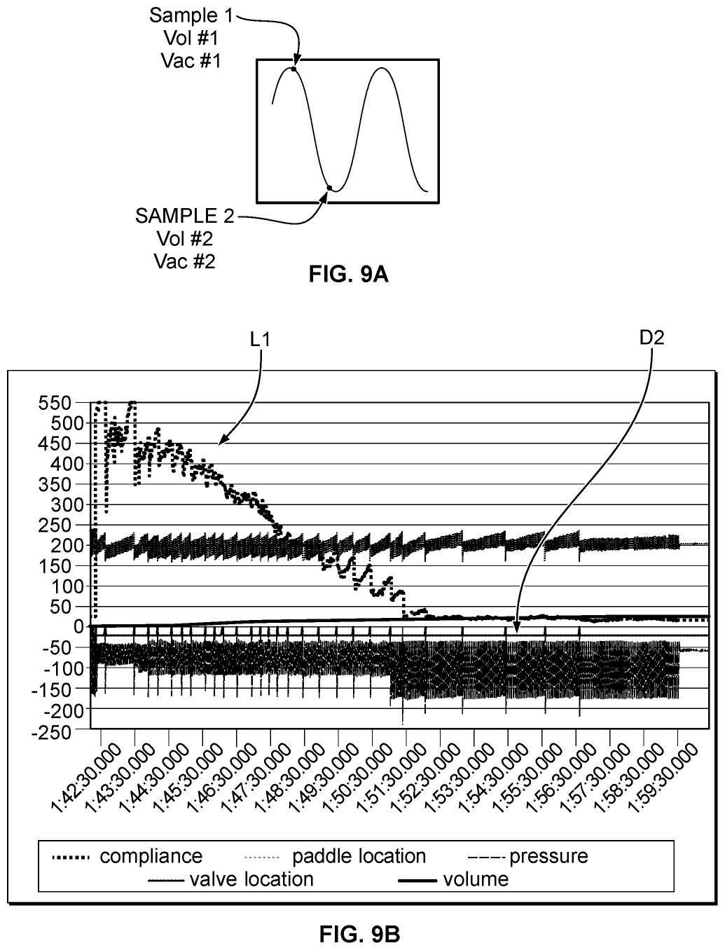

This data is acquired by sampling using all the existing rules of the map, but when the pinch foot is open during normal pumping, in the course of a waveform. A sample is taken near to the top of the waveform and near to the bottom to facilitate separating the samples in volume and in vacuum, which in turn provides less noise on in the ratio. However, samples could be made at any two points in the waveform that meet the good sampling practices of vacuum system and the volume map. Shown in FIG. 9A is an example where on a vacuum waveform one might choose to sample for best results. Good sampling practices require minimum forces needed for the map to be accurate and always ensuring the motor contains no backlash (motor has recently been moving out). In one approach, a sample is taken initially after the wave has started moving out, and later shortly before it turns around.

With reference to FIG. 9B, there is shown pump data taken from a pump as it fills with milk. A descending line L1 represents the measurement of a change in volume relative to a change in vacuum times k, where k is a constant that is included to make the signal easier to visualize. This line L1 drops as the system fills with fluid, and is used as a signal to the system to identify air and fluid in the system, such as when the system is full of milk. In this particular example, the system was determined to be full of milk at time 1:51:00 as reflected in the sudden rise in vacuum level as represented by the bottom data representation D2. Once detected, users are alerted through an action of the pump and/or a message sent to an auxiliary computer device such as a smartphone.

These data is also used for leak detection. For example, where line L1 ceases to continue to drop while the pump continues purging, an air leak is detected. Moreover, a compliance measurement that first drops and then rises later is an indication of an air leak that started later in a session.

In an alternative approach, changes in how compliance is determined to minimize the effect of flow rate on compliance measurement is provided. Here, the approach minimizes or eliminates flow rate as a factor in compliance to indicate when the conduit fills with milk and/or to determine if there is structural damage in the conduit or pump hardware, or if there is an air leak or a misalignment. The change in volume to change in vacuum calculation is the same but the samples are taken at different points in the waveform. That is, whereas the samples are taken in the immediately preceding described approach when the phase of a vacuum waveform is increasing from a minimum to a maximum, samples in this approach are taken when the phase of a waveform vacuum is decreasing from a maximum to a minimum. Accordingly, rather than the first sample point being at a low vacuum part of the wave and the second sample point being at a high vacuum of the wave, the samples are taken in reverse; the first sample point being at the high vacuum part of the wave and the second sample being at the low vacuum part of the wave. This takes advantage of the ability for a pump system to easily achieve a low vacuum target and is useful in systems where reaching a maximum vacuum is challenging. Consequently, detecting when a system is full of fluid can be accomplished without or with less regard to fluid flow rate since taking the first sample point at a high vacuum part of a wave offers more stability at different fluid flow rates, during changes of waveform shape, and at slow or high frequency and waveform amplitude. Moreover, in this approach when vacuum is decreasing, compliance is approximately the same regardless of fluid flow rate, both when the conduit is empty or full and thus, conduit volume prior to when it is full can be estimated with enhanced accuracy.

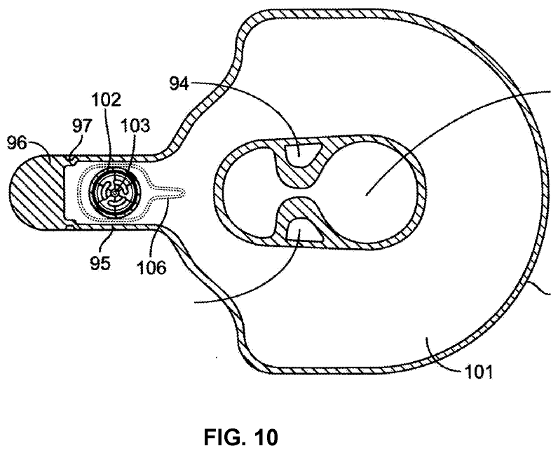

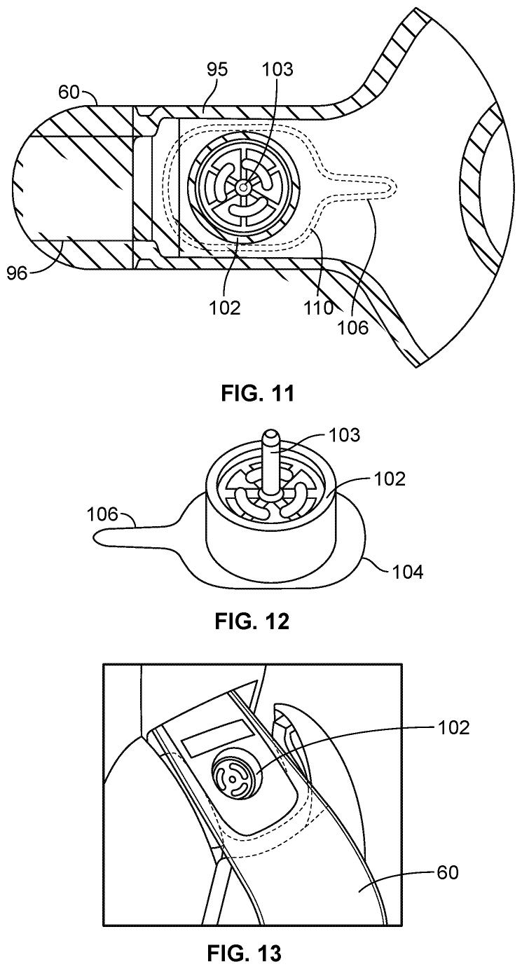

Turning now to FIGS. 10-13, one embodiment of a collection or container assembly 60 is shown. In one particular embodiment, the collection or container assembly 60 can be formed from two 2.5-3.0 mil sheets of material that can be band welded or otherwise joined together along a perimeter 92 of the assembly, and can be sized to retain 3.5 ounces or more or up to 4.5 ounces, or alternatively 8 ounces of fluid. In particular, the collection or container assembly 60 can be pre-formed to optimize or maximize the space inside the pump system and flange. For shipping, the collection or container assembly can be pulled closed with a vacuum to make it flat or thin for packaging or handling. A body of the collection or container assembly is generally bladder shaped and includes a generally central opening 93 created by an interior band seal. In one particular approach, the body can additionally include gussets to provide more volume. A pair of wings 94 could extend into the central opening 93 and are provided for handling and facilitating positioning of the collection or container assembly 60 within a pump system 10. A narrow neck portion 95 is centrally positioned and extends longitudinally away from the central opening 93. The neck portion 95 includes a tab portion 96 that provides structure for grasping and removal, and can further include one or more cut-outs or tear-able elements 97 provided for aiding in tearing the container 90. Further scoring is also contemplated to help in the tearing of the bag assembly 90. Also, in alternative embodiments, the collection or container assembly 90 can be re-sealable, re-usable, include larger or smaller openings or include spout structure for pouring contents. A spout can also be attached to the fitment or valve of the collection assembly or otherwise formed in the container to facilitate pouring. Such a spout could further include structure which temporarily or permanently defeats the valve or fitment. The valve of the collection or container assembly can also be re-usable with a second or subsequent collection or container assembly, and therefore is removable from the container assembly.

It is contemplated that the system is configured to pump into a sealed collection or container assembly 60, or one that includes an integral valve or an otherwise airtight collection or container assembly 60, or combinations thereof. In this specific regard, the system can alternatively or additionally be closed and never vented to the atmosphere, and/or the system suction is only reduced through the flow of milk into the system. Thus, in at least one approach, milk or fluid that is pumped through the system is never exposed to new outside air from the environment once it enters the collection or container assembly. Accordingly, the orientation of the pump system or person has virtually no impact on the functioning of the system (i.e., no spills). The collection or container assembly can include a rigid or flexible sealing component, such as a ring or gasket into which the pump or container valve is pushed or twisted and sealed. The collection or container assembly can also include an opening or hole or structure that is pierced such that the container assembly seals about the member that goes into it. Moreover, there are contemplated a range of disposable and durable combinations of container 101 and valve fitment 102 arrangements such that one or both of the container bag 101 and fitment 102 are disposable or reusable. Additionally, the container can be configured to be inside or outside of the pump housing.

The fitment 102 can embody a valve such as an umbrella valve assembly 103 or other type of one-way valve connected in fluid communication with the storage container 101. The fitment can also assume a myriad of alternative embodiments, and can additionally or alternatively be formed integral with the container. For example, in one contemplated approach, the fitment and/or the valve can be formed as part of the container rather than define a separate component attached to the container. As shown in FIGS. 8-10, however, the tail 104 of the umbrella valve 103 can be employed to defeat the valve when desired such as to remove gases, by turning it and engaging the tail against the valve body. Additionally, the valve includes a generally cylindrical portion having a diameter of approximately 0.585 inches extending from a flat base 104 having a width of approximately 0.875 inches. It is the flat base portion 104 that is captured and sealed between the two sheets of bag container material and includes a tail 106. The tail 106 functions to ensure flow through the neck portion of the container assembly 60 particularly when it is placed into the pump assembly (See FIG. 12), and has a narrow, elongated shape that permits flow thereabout. That is, the tail 106 maintains flow through the neck even when the neck is folded as the container assembly is attached to the breast pump body. Valve 103 prevents back flow of milk into the flex-tube 32, and facilitates maintaining the suction (vacuum) level in the flex-tube 32. In other embodiments, other features can be provided or built into a valve to allow for depression or otherwise overcome the valve to vent air. Such approaches can involve a protrusion that is attached or associated with the valve so that as the protrusion is pushed toward the collection or container assembly, an edge of the valve is translated to thereby break the valve internal seal. Moreover, a nub can be attached to valve structure and configured inside the container assembly. Tugging on the nub through a layer of the container assembly thus results in freeing an edge of the valve and breaking the valve seal.

In at least one embodiment, the pressure at which the valve 103 opens to allow flow into the milk collection container 60 is about 25 mm Hg. The valve 103 can be configured and designed such that it allows fluid to flow through it when the pressure in conduit or flex tubing 32 is positive, e.g., about 25 mm Hg, or some other predesigned “crack pressure”. The action of the compression elements cycles between increasing vacuum when the compression elements move in a direction away from flex-tube 32 and decreasing when the compression elements compress the flex-tube 32, but typically should not increase the vacuum to greater than the predetermined maximum vacuum. As the compression elements 36, 38 compress the flex-tube 32, the pressure in the system 10 goes up and reaches the minimum suction level (e.g., latch suction level, such as −60 mmHg, −30 mm Hg, or some other predetermined latch suction level), at which time the compression member (pinch valve) 36 seals off portion 32S thereby maintaining the minimum suction (latch suction) against the breast. Continued compression of portion 32L by compression member 38 continues to increase the pressure downstream of compression member 36, until the crack pressure is reached (e.g., 25 mm Hg or some other predetermined, positive crack pressure), that opens the valve 103. The compression elements 36, 38 continue compressing flex-tube 32, pumping fluid (milk) through the valve 103 and into the collection container assembly 60 until the compression element 38 reaches an end point in travel. The end point in travel of the compression element 38 against portion 32L may be predetermined, or may be calculated in real time by the controller 52 using feedback from pressure sensor 54 and feedback from the driver of the compression element 38, from which the controller 52 can calculate the relative position of the compression element 38 over the course of its travel. The compression member 36 remains closed throughout this process, as it is used to seal off the tube 32 for a necessary time that the compression element 38 is pumping milk out into the collection container assembly 60. As the compression elements 36, 38 reverse direction and pull away from the flex-tube 32, they start the cycle again.

As milk enters the system, the suction level decreases (pressure increases). The feedback provided by pressure monitoring via pressure sensor 54 provides input to a feedback loop that adjusts the position of the compression member 38 to maintain the desired vacuum (pressure) within the conduit or flex tubing 32 by compensating for the changes in pressure that occur to changing amounts of milk in the flex tubing 32.

As the pump system 10 goes through a power up routine, the controller 52 reads force on the load cell when a load cell is used as the pressure sensor 54. This is the load measured by the load cell, before the skin contact member 14 has been applied to the breast, so in one approach it is a state in which the pressure in the conduit or flex-tube 32 is atmospheric pressure. The controller 52 then calibrates the system such that the preload force or position or measured load or strain equates to atmospheric pressure. Based upon a neural network or computer learning, load or strain detected at the flex-tube 32 can be converted to pressure readings in the system 10 during operation of the breast pump system 10 upon attachment to the breast.

The system 10 can calculate the volume of milk pumped into system or alternatively the volume collected in the milk collection container assembly 60 in the manner described above. When it is determined that the milk collection container is full, the pumping will cease. An override can be incorporated into the system so that the user can choose to continue pumping beyond normal full bag detection. By knowing the dimensions of the conduit or flex tubing 32 downstream of the compression member 36 when compression member 36 has sealed off tubing portion 32S, the overall volume capacity of the system 10 downstream of compression member 36 can be calculated. With reference again to FIG. 7, tracking of the position of the compression member 38 relative to the tube 32 (such as by knowing the motor driver 46 position at all times, for example via an encoder), dictates the volume change in the tubing 32. As the pumping process is carried out, pumping/purging of milk into the milk collection container occurs when the compression member 36 has closed off the small tube portion 32S at the location of compression. When the compression member 36 has closed off tube portion 32S, the change in position of compression member 38 that occurs to carry out the purge of milk from the flex tubing 32 and into the milk collection container 60 is used to calculate the change in volume of the tubing 32 downstream of the compression member 36, which equates with volume of milk and/or air that is pushed into the milk collection container 60 bag.

The number of purges can be tracked when the system is full for the purpose of measuring flow. As stated, it can be determined when the system 10 is purging fluid versus purging air since the forces are much higher for purging fluid than purging air. Thus, counting the number of purges that contain fluid, and knowing the volume that is purged for each purge leads to a calculation of flow without requiring significant system tuning or calibration, and avoiding confusing a slow air leak with flow. Leaks can also be detected by employing an algorithm involving closing the pinch compression member, followed by closing the pump compression or paddle member, and then pulling the pump compression member outwardly to create a vacuum, or alternatively separate from a purge by measuring, moving a paddle and measuring again. By then holding the pump compression member in this position and verifying the vacuum is maintained, it can be determined if there is a leak in the system 10.

In addition to calculating the volume of milk purged with each purge cycle, the system (via controller 52) can sum the volumes from all purge cycles to calculate the total volume entering the pump or alternatively pushed into the milk collection container 60 during a milk extraction session. This volume can be stored with a unique identifier provided to the milk container so that the system 10 keeps a record of how much milk is stored in each milk collection container 60. This information can also be time stamped so that the user will know the time and date that milk was collected, regarding each milk collection container. Additional statistics can be calculated, including, but not limited to: average volume per extraction session, total volume extracted for any given day, average milk extraction volume per day, etc. Any and all of this data can be exported to an external computer, either manually, or it may be automatically uploaded to the computer when the computer is within range of the system 10 for wireless communication, or when the computer is connected to the system by wire. One value is thus communicating to the user which milk to use first, which is expiring and how much the user has stored. Further optionally, any or all of this data can be either manually or automatically uploaded to a cloud service over the Internet, either wirelessly or by wire.

When a user has completed the pumping phase of extracting milk from a breast, it is useful and efficient to purge as much milk that remains in the tubing 32 from the tubing 32 and into the milk collection container 60. Ending of the extraction phase can be performed upon elapse of a predetermined extraction phase time, calculation of a predetermined amount of milk having been pumped, manual cessation of the extraction phase by the operator, or some other predetermined value having been achieved after performing the extraction. The direction of the pumping stroke of compression member 38 is reversed and the compression member 38 is run in the reverse direction to decrease suction within the tubing 32 and optionally create a small positive pressure within the tubing 32 to facilitate removal of the system 10 from the breast. Alternatively, the suction may be decreased to a level where a slight suction remains so that the user still pulls the system 10 of the breast to detach it. Possibly the vacuum is reduced to 0 mmHg, or a slightly positive pressure to automatically detach the system 10 from the breast. The end pressure value where the pressure reduction by reverse pumping is ceased can be in the range of about −60 mmHG (weak vacuum) to a positive 50 mmHg (e.g., the crack pressure of the valve to the container). The compression member 36 does not close off the tubing portion 32S during this process, rather, tubing portion 32S remains open. Initiation of this reverse pumping may occur automatically or, alternatively, may be initiated by the user. This process continues until the seal of the system 10 to the breast is broken, which is detected by the controller via sensor 54. Once exposure of the tubing 32 to atmospheric pressure is detected, the stroke direction of pumping is again reversed thereby pumping the milk in tubing 32 under positive pressure and driving the milk from the tubing 32 into the container 60. If by chance, the system 10 accidentally or otherwise becomes resealed to the breast during purge pumping, and the user does not wish to pump, the system 10 can automatically shut down as it senses vacuum pressure being regenerated in the vicinity of the flange or breast/skin contact member 14. Where there is not a clear indication that the user does not wish to pump, then the system will assume that pumping is desired and will not shut down automatically.

The system 10 can be configured to distinguish whether it has been attached to the left breast or the right breast of the user. This can be useful for tracking milk volume output per breast, per session, total daily volume per breast, etc. When using two of the pump systems, the tracking of data for each breast can still be maintained accurately, even when one of the pump systems 10 is attached to the left breast during a current pumping session after having been attached to the right breast during a previous pumping session. In one embodiment, the pumping systems 10 can establish current location (i.e., left or right breast) by receiving a signal from the other pumping system having been attached to the other breast. This established relative left-right locations of the two pumping systems 10, so that each system 10 can accurately record as to whether milk is being extracted from the right breast or left breast. This identification is automatic, without any user input required and it also relieves the burden on the user to otherwise keep track of which pump system 10 is placed on each breast and to maintain this order with each successive pumping session. Left and right pump labeling is also contemplated such as by placing markings on the system housing or cover jack, for example, near the power cover. Stickers or other markings could be given to customers with their device to help differentiate between right and left.

The system 10 can calculate the pressure during operation in any of the manners described above. The suction (pressure) level can be varied as desired, and by continuously or repeatedly measuring/calculating pressure, the feedback provided by sensor(s) 54 to controller 52 provides a control loop that can be used to adjust the compression member 38 position and/or speed to vary the suction pressure to a level desired, or maintain a desired suction pressure in real time. Thus, controller 52 can control the positions and speeds of compression members 36, 38 to achieve any vacuum pressure pumping profile desired, and provide automatic, real time adjustments to maintain a desired vacuum pressure within the system. Also contemplated is responding in real time to maintain flow. This can be accomplished independent or in conjunction with monitoring and regulating pressure in real time.

The controller 52 tracks the position of the compression member 38 relative to the tubing 32L, such as by keeping track of the driver 46 position or shaft position (interconnecting linkage between driver 46 and compression member 38), and calculates (or looks up) pressure based upon data received from sensor 54. The system controller or firmware is programmed with or retains information relating values detected by system sensors with driver positions and speed and system pressure. Thus, changes in position and/or speed of the compression member 38 by controller 52 can be controlled by resulting changes in pressure calculated or looked up, relative to the pressure sought to be achieved. As stated above, by using machine learning or supervised learning regression techniques, the system 10 can be trained to interpret the motor positioning and tubing strain (as well as motor speed or pump settings), while compensating for noise and hysteresis, to arrive at a pressure/vacuum level. More specifically, a neural net system or other mathematical regression can be incorporated into system firmware so that sensor input can be translated to pressure/vacuum levels. Controller 52 can thus control compression member 36 in a similar manner, but control of member 36 is more focused on position control, as the compression member 36 needs to fully close off tube portion 32S when maintaining latch suction against the breast/nipple. However, the closing off is timed and performed at the determined latch pressure, which is known from the data received from sensor 54.

While the present disclosure has been described with reference to the specific embodiments thereof, it should be understood by those skilled in the art that various changes may be made and equivalents may be substituted without departing from the true spirit and scope of the disclosure. In addition, many modifications may be made to adapt a particular situation, material, composition of matter, process, process step or steps, to the objective, spirit and scope of the present disclosure. All such modifications are intended to be within the scope of the present disclosure.

That which is claimed is:

1. A wearable system to pump fluid from a breast, the system comprising:
a skin contacting structure configured and dimensioned to form a seal with the breast;
a pump that provides a suction within the skin contacting structure;
a pathway through which fluid is pumped, the pathway capable of including a closed segment; and
a controller that automatically calculates volumes pumped through the closed segment.

2. The system of claim 1, further comprising a strain gauge sensor and a motor position sensor, and a map correlating strain gauge sensor and motor sensor measurements to volumes pumped through the closed segment.

3. The system of claim 1, wherein the wearable system maintains at least a latch suction throughout a pumping cycle.

4. The system of claim 1, wherein the controller is configured to control operational settings of the wearable system.

5. The system of claim 1, wherein the system takes measurements before and after a purge, the difference between volume measurements enable a total volume purged to be determined of air and fluid.

6. The system of claim 1, wherein the controller is configured to adjust pumping in real time.

7. The system of claim 1, further comprising a compression member that closes the pathway at one end and a valve that is closed at another end of the pathway.

8. The system of claim 1, wherein the controller optimizes pumping by adjusting pump settings.

9. The system of claim 1, wherein the controller adjusts pump settings to be correlated with the comfort of the pump sessions based on feedback.

10. The system of claim 1, further comprising a flange, a chassis and a housing, wherein the flange, chassis and housing assemble together.

11. The system of claim 1, wherein the controller makes pump adjustments, and pumping settings are tracked.

12. The system of claim 1, wherein the controller controls a pumping function and modifying pumping to reach targets in real time.

13. The system of claim 1, wherein the system is configured to store a variety of pump settings.

14. The system of claim 1, wherein pumped volume of fluid or air measurements are taken before and after a purge.

15. The system of claim 1, wherein from multiple purges in succession allows for continuous air leaks to be detected, and accurate cumulative volume of air and fluid pumped into the milk receptacle to be calculated.

16. The system of claim 1, further comprising a collection assembly that is placed within an interior of the system.

17. The system of claim 1, wherein a total volume purged is determined when calculating differences in measurements before and after a purge.

18. The system of claim 1, wherein a combination of multiple measurements each before and after purges enable a determination of total volume of air expelled and total volume of fluid expelled in a purge.

19. The system of claim 1, wherein volume determinations accommodate for motor or flextube component variabilities.

20. The system of claim 1, wherein measurements of volume and vacuum differential during pumping are used in real time to determine air content, or whether there is an air leak to the pump septum.
